(19) United States
(12) Patent Application Publication
(10) Pub. No.: US 2004/0077576 A1
(43) Pub. Date: Apr. 22, 2004

(54) Minimal plasmid vectors that provide for persistent and high level gene expression and methods for using the same

(76) Inventors: Mark A. Kay, Los Altos, CA (US); Nakai Hiroyuki, Redwood City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/642,362

(22) Filed: Aug. 14, 2003

Related U.S. Application Data
(63) Continuation-in-part of application No. PCT/US02/04975, filed on Feb. 15, 2002.
(60) Provisional application No. 60/269,607, filed on Feb. 16, 2001.

(51) Int. Cl. A61K 48/00

(52) U.S. Cl. 514/44; 435/455

(57) ABSTRACT

Methods are provided for the in vivo introduction of an expression cassette into a target cell of a vascularized organism, e.g., a mammal, in manner such that the encoded protein of the introduced expression cassette is persistently expressed at a high level in the target cell. In the subject methods, an aqueous formulation of a minimal plasmid vector that includes the expression cassette is administered into the vascular system of the organism. The minimal plasmid vector employed in the subject methods is one that provides for persistent and high level expression of an expression cassette that is present on the vector in a manner that is substantially expression cassette sequence and direction independent Also provided are the minimal plasmid vectors employed in the subject methods. The subject methods and compositions find use in a variety of different applications, including both research and therapeutic applications, and are particularly suited for use in the in vivo delivery of nucleic acids encoding protein products, particularly where persistent, high level protein expression is desired without integration of the vector into the host genome.

### CROSS-REFERENCE TO RELATED APPLICATIONS

This application is a continuation-in-part application of application serial no. International Application No. PCT/US02/04975, which application (pursuant to 35 U.S.C. § 119 (e)) claims priority to the filing date of the U.S. Provisional Patent Application Serial No. 60/269,607 filed Feb. 16, 2001; the disclosures of which are herein incorporated by reference.

### INTRODUCTION

1. Field of the Invention

The field of this invention is molecular biology, particularly transformation and specifically vectors employed in transformation.

2. Background of the Invention

The introduction of an exogenous nucleic acid sequence (e.g., DNA) into a cell, a process known as “transformation,” plays a major role in a variety of biotechnology and related applications, including research, synthetic and therapeutic applications. Research applications in which transformation plays a critical role include the production of transgenic cells and animals. Synthetic applications in which transformation plays a critical role include the production of peptides and proteins. Therapeutic applications in which transformation plays a key role include gene therapy applications. Because of the prevalent role transformation plays in the above and other applications, a variety of different transformation protocols have been developed.

In many transformation applications, it is desirable to introduce the exogenous DNA in a manner such that it provides for long-term expression of the protein encoded by the exogenous DNA. Long-term expression of exogenous DNA is primarily achieved through incorporation of the exogenous DNA into a target cell's genome. One means of providing for genome integration is to employ a vector that is capable of homologous recombination. Techniques that rely on homologous recombination can be disadvantageous in that the necessary homologies may not always exist; the recombination events may be slow; etc. As such, homologous recombination based protocols are not entirely satisfactory.

Accordingly, alternative viral based transformation protocols have been developed, in which a viral vector is employed to introduce exogenous DNA into a cell and then subsequently integrate the introduced DNA into the target cell's genome. Viral based vectors finding use include retroviral vectors, e.g., Moloney murine leukemia viral based vectors. Other viral based vectors that find use include adenovirus derived vectors, HSV derived vectors, sindbis derived vectors, etc. While viral vectors provide for a number of advantages, their use is not optimal in many situations. Disadvantages associated with viral based vectors include immunogenicity, viral based complications, and the like.

Therefore, there is continued interest in the development of additional methods of transforming cells with exogenous nucleic acids to provide for persistent, long-term expression of an encoded protein. Of particular interest is the development of a non-viral in vivo nucleic acid transfer protocol and vector that provides for persistent protein expression without concomitant genome integration, where the vector provides for persistent expression in a manner that is independent of the sequence and direction of the of the expression cassette present on the vector.

Relevant Literature

U.S. Pat. Nos. of interest include 5,985,847 and 5,922,687. Also of interest is WO/I 1092. Additional references of interest include: Wolff et al., “Direct Gene Transfer Into Mouse Muscle In Vivo,” Science (March 1990) 247:1465-1468; Hickman et al., “Gene Expression Following Direct Injection of DNA Into Liver,” Hum. Gen. Ther. (December 1994) 5:1477-1483; and Acsadi et al., “Direct Gene Transfer and Expression Into Rat Heart In Vivo,” New Biol. (January 1991) 3:71-81.

### SUMMARY OF THE INVENTION

Methods are provided for the in vivo introduction of an expression cassette into a target cell of a vascularized organism, e.g., a mammal, in manner such that the encoded protein of the introduced expression cassette is persistently expressed at a high level in the target cell. In the subject methods, an aqueous formulation of a minimal plasmid vector that includes the expression cassette is administered into the vascular system of the organism. The minimal plasmid vector employed in the subject methods is one that provides for persistent and high level expression of an expression cassette encoded product that is present on the vector in a manner that is substantially expression cassette sequence and direction independent. Also provided are the minimal plasmid vectors employed in the subject methods. The subject methods and compositions find use in a variety of different applications, including both research and therapeutic applications, and are particularly suited for use in the in vivo delivery of nucleic acids encoding protein products, particularly where persistent, high level protein expression is desired without integration of the vector into the host genome.

### BRIEF DESCRIPTION OF THE DRAWINGS

FIG. 1 provides a schematic view of representative minimal plasmid vector backbones according to the present invention.

FIG. 2 hFIX levels in mouse plasma following high-pressure tail vein injection of HFIX plasmid vectors. Six to eight-week old C57B1/6 mice received 25 μg of supercoiled closed circular plasmid, pNEF1αhFIX For, pNEF1αhFIX Rev, or linearized plasmid, pNEF1αhFIX Lin. pNEF1αhFIX For and pNEF1αhFIX Rev carry the same components (pN backbone and the EF1α-hFIX expression cassette) but the pN backbone is placed in different orientations, i.e., in the same orientation relative to the expression cassette for pNEF1αhFIX For while in the opposite orientation for pNEF1αhFIX Rev. pNEF1αhFIX Lin is the mixture of linearized pN backbone and EF1α-hFIX expression cassette.

FIGS. 3 and 4 Comparison of the effect of pBS and pN backbones on persistent hFIX expression from mouse hepatocytes transduced by hFIX plasmid vectors. Six to eight-week old C57B1/6 mice received 25 μg (for pN constructs) or 30 μg (for pBS constructs) of hFIX plasmid vector by high-pressure tail vein injection, and plasma hFIX levels were followed.

CM1 is a liver-specific promoter-driven hFIX expression cassette. TEF1αhFIX is an EF1α-hFIX expression cassette with Sleeping Beauty transposon inverted terminals.

FIG. 5 Determination of the plasmid backbone element responsible for position and orientation-dependent inhibitory effect. (A) hFIX levels in mouse plasma 6 weeks after high-pressure tail vein injection of EF1α0 hFIX plasmid vectors carrying a series of minimal plasmid backbones. The numbers 1 to 14 below each bar represent each plasmid backbone number as indicated in FIG. 5C. Bars 15 to 17 represent the values from linearized plasmids carrying pN1, pBS and pNkan respectively. For 15 to 17, the mixture of linearized plasmid backbone and EF1α-hFIX expression cassette were injected. (B) HFIX levels in mouse plasma 8 weeks after high-pressure tail vein injection of CM1 plasmid vectors carrying a series of minimal plasmid backbones. Bar 18 represents the value from linearized pN1CM1 plasmid (or the mixture of linearized pN1 backbone and CM1 cassette). (C) A schematic view of pN backbone family. pN1 to pN4 carry essentially the same sequence but position and orientation of each element relative to the promoter (P) of transgene is different with each other. pN5, pBS and pNKan are as described in FIG. 1.

FIG. 6 Plasmid backbone size effect to transgene expression. Various sizes of DNA fragments from KanR gene (0-500 bp, 50 bp increments) were inserted between the expression cassette and AmpR in pN5EF1αhFIX For or Rev constructs. hFIX levels in mouse plasma 18 weeks after high-pressure tail vein injection of these plasmids are shown. The size of the plasmid backbone dramatically affected the transgene expression.

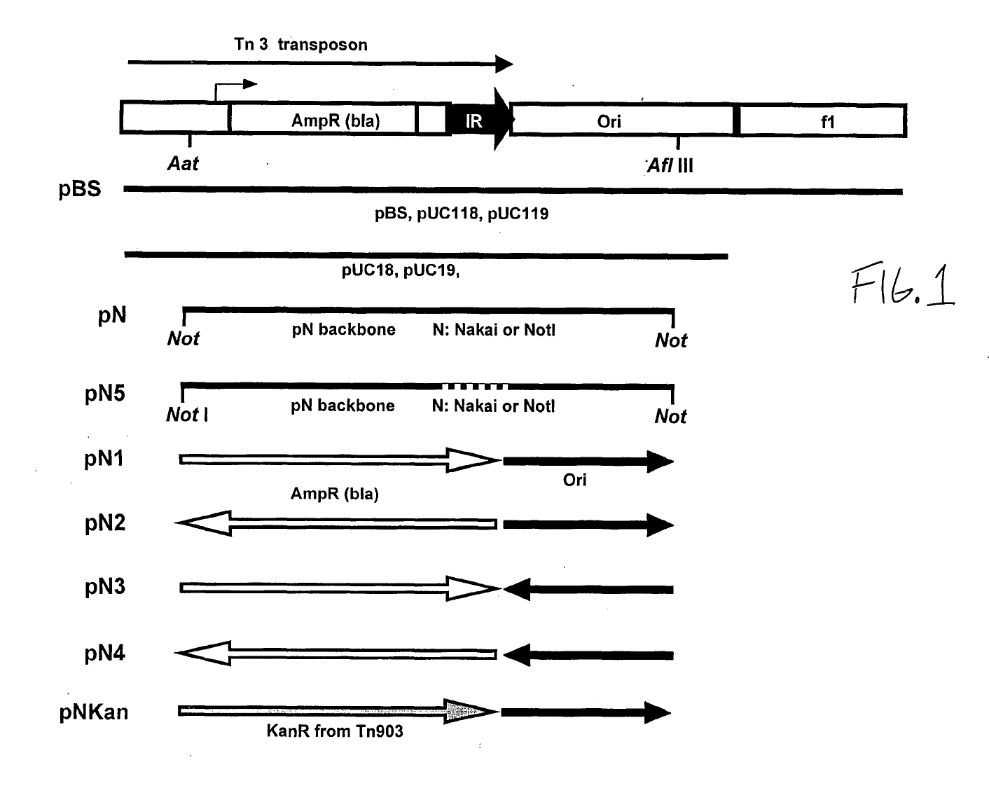

### DESCRIPTION OF THE SPECIFIC EMBODIMENTS

Methods are provided for the in vivo introduction of an expression cassette into a target cell of a vascularized organism, e.g., a mammal, in manner such that the encoded protein of the introduced expression cassette is persistently expressed at a high level in the target cell. In the subject methods, an aqueous formulation of a minimal plasmid vector that includes the expression cassette is administered into the vascular system of the organism. The minimal plasmid vector employed in the subject methods is one that provides for persistent and high level expression of an expression cassette that is present on the vector in a manner that is substantially expression cassette sequence and direction independent. Also provided are the minimal plasmid vectors employed in the subject methods. The subject methods and compositions find use in a variety of different applications, including both research and therapeutic applications, and are particularly suited for use in the in vivo delivery of nucleic acids encoding protein products, particularly where persistent, high level protein expression is desired without integration of the vector into the host genome.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the cell lines, vectors, methodologies and other invention components that are described in the publications which might be used in connection with the presently described invention.

### Methods

In the broadest sense, the present invention provides methods of introducing an exogenous nucleic acid into at least the nucleus of at least one cell, i.e., a target cell, of a multicellular organism. In many embodiments, the present invention provides methods of introducing an exogenous nucleic acid into the nucleus of a plurality of the cells of the host, whereby plurality is often meant at least about 0.1 number %, usually at least about 0.5 number % in certain embodiments. A feature of the subject invention is that the subject methods are in vivo methods, by which is meant that the exogenous nucleic acid is administered directly to the multicellular organism, in contrast to in vitro methods in which the target cell or cells are removed from the multicellular organism and then contacted with the exogenous nucleic acid. As specified below, in many embodiments the subject methods rely on systemic administration of the vector employed in the subject methods, where by systemic administration is meant that the vector is administered to the host in a manner such that it comes into contact with more than just a local area or region of the host, where by local area or region of the host is meant a region that is less than about 10%, usually less than about 5% of the total mass of the host. In other embodiments, local administration protocols are employed. While in the broadest sense the subject methods are methods of introducing any nucleic acid into a host, generally, the exogenous nucleic acid is an expression cassette that encodes a product, e.g., protein, of interest, as described in greater detail infra.

Minimal Plasmid Vector

A feature of the subject invention is that the methods employ a minimal vector to deliver the exogenous nucleic acid, hereinafter referred to as “expression cassette” for convenience, to the target cell or cells of the host. The minimal vector employed in the subject methods is a plasmid vector, i.e., it is a double-stranded circular DNA molecule. The sequence of the plasmid vector employed in the subject methods is such that it provides for persistent, high level expression of an expression cassette encoded protein present on the vector in a manner that is at least substantially expression cassette sequence and direction independent.

As summarized directly above, a feature of the subject minimal vectors is that they provide for persistent expression of the expression cassette encoded protein present thereon, as opposed to transient or short-lived expression. By persistent expression is meant that the expression of encoded product, e.g., protein, at a detectable level persists for an extended period of time, if not indefinitely, following administration of the subject vector. By extended period of time is meant at least 1 week, usually at least 2 months and more usually at least 6 months. By detectable level is meant that the expression of the encoded product is at a level such that one can detect the encoded product in the mammal, e.g., in the serum of the mammal, at a therapeutic concentration. See e.g., the experimental section, supra. As compared to a control in which the pBluescript plasmid vector (Stratagene Corporation, La Jolla, Calif.) is employed, protein expression persists for a period of time at a detectable level that is at least about 2 fold, usually at least about 5 fold and more usually at least about 10 fold longer following the subject methods as compared to a control. An encoded product is considered to be at a detectable level if it can be detected using technology and protocols readily available and well known to those of skill in the art. The experimental section infra provides representative detectable levels of the human factor IX protein in mouse serum.

Typically, the above described persistent expression is not only at a detectable level, but at a high level. A minimal vector is considered to provide for a high level of expression if, after a period of time following its administration, e.g., at least about 28 days, the protein encoded by the expression cassette of the vector is present at high levels in the host, e.g., in the target cells, in the serum of the host, etc. Levels of an encoded product are considered “high” for purposes of the present application if they are present in amounts such that they exhibit detectable activity (e.g., have an impact on the phenotype), e.g., therapeutic activity, in the host. Whether or not the expression level of a particular product is high will necessarily vary depending on the nature of the particular product, but can readily be determined by those of skill in the art, e.g., by an evaluation of whether expression of the product is sufficient to exhibit a desired effect on the phenotype of the host, such as an amelioration of a disease symptom, e.g., reducing clotting time, etc. A minimal plasmid can be tested to see if it provides for the requisite high level of protein expression by administering it to a host according to the protocols described, infra, and testing for the desired expression level, e.g., in the blood or serum where the expression protein is secreted from the target cell where it is produced, in a tissue lysate of the target cells for non-secreted proteins, and the like.

The minimal plasmid vectors employed in the subject invention provide for the above described persistent, high level expression in a manner that is substantially expression cassette sequence and direction independent. By expression cassette sequence and direction independent is meant that the expression manner, e.g., persistent high level expression, of the expression cassette encoded protein does not substantially vary regardless of the particular sequence of the expression cassette or the direction of the expression cassette in the minimal plasmid vector. Expression cassette sequence refers to the nucleic acid sequence of the expression cassette while expression cassette direction refers to the orientation of the expression cassette elements on the plasmid vector. As the subject minimal plasmid vectors have a sequence that makes them substantially expression cassette sequence and direction independent, any variation observed in the expression profile achieved in a particular vector between any two different expression cassettes, which may differ from each other in terms of sequence and/or direction, will not vary by more than about 20%, usually not more than about 10% and more usually not more than about 5%, where this variation value is modified to account for variations in different promoters, cellular environments etc., which may influence the expression level of the expression cassette independent of the minimal plasmid vector. Variation is typically determined in terms of detected encoded product expression level in the host. A particular minimal plasmid vector can be readily determined by those of skill in the art to be expression cassette sequence and direction independent by employing the protocol used to evaluate the representative minimal plasmid vectors described in the experimental section, infra.

The minimal plasmid vectors employed in the subject methods include several elements that provide for their utility in the subject methods. The subject minimal plasmid vectors include at least one restriction endonuclease recognized site, i.e., a restriction site. A variety of restriction sites are known in the art and may be included in the vector, where such sites include those recognized by the following restriction enzymes: HindIII, PstI, SalI, AccI, HincII, XbaI, BamHI, SmaI, XmaI, KpnI, SacI, EcoRI, and the like. In many embodiments, the vector includes a polylinker, i.e., a closely arranged series or array of sites recognized by a plurality of different restriction enzymes, such as those listed above. As such, in many embodiments, the vectors include a multiple cloning site made up of a plurality of restriction sites. The number of restriction sites in the multiple cloning site may vary, ranging anywhere from 2 to 15 or more, usually 2 to 10.

When employed, the minimal plasmid vectors typically include at least one nucleic acid of interest, i.e., a nucleic acid that is to be introduced into the target cell, e.g., to be expressed as protein in the target cell, etc., as described in greater detail below, where the nucleic acid is typically present as an expression cassette. The subject vectors may include a wide variety of nucleic acids, where the nucleic acids may include a sequence of bases that is endogenous and/or exogenous to the multicellular organism, where an exogenous sequence is one that is not present in the target cell while an endogenous sequence is one that pre-exists in the target cell prior to introduction. In any event, the nucleic acid of the vector is exogenous to the target cell, since it originates at a source other than the target cell and is introduced into the cell by the subject methods, as described infra. The nature of the nucleic acid will vary depending the particular protocol being performed. For example, in research applications the exogenous nucleic acid may be a novel gene whose protein product is not well characterized. In such applications, the vector is employed to stably introduce the gene into the target cell and observe changes in the cell phenotype in order to characterize the gene. Alternatively, in protein synthesis applications, the exogenous nucleic acid encodes a protein of interest which is to be produced by the cell. In yet other embodiments where the vector is employed, e.g., in gene therapy, the exogenous nucleic acid is a gene having therapeutic activity, i.e., a gene that encodes a product of therapeutic utility.

A variety of different features may be present in the vector. In many embodiments, the vector is characterized by the presence of at least one transcriptionally active gene. By transcriptionally active gene is meant a coding sequence that is capable of being expressed under intracellular conditions, e.g., a coding sequence in combination with any requisite expression regulatory elements that are required for expression in the intracellular environment of the target cell into which the vector is introduced by the subject methods. As such, the transcriptionally active genes of the subject vectors typically include a stretch of nucleotides or domain, i.e., expression module or expression cassette, that includes a coding sequence of nucleotides in operational combination, i.e. operably linked, with requisite transcriptional mediation or regulatory element(s). Requisite transcriptional mediation elements that may be present in the expression module include promoters, enhancers, termination and polyadenylation signal elements, splicing signal elements, and the like.

Preferably, the expression module or expression cassette includes transcription regulatory elements that provide for expression of the gene in a broad host range. A variety of such combinations are known, where specific transcription regulatory elements include: SV40 elements, as described in Dijkema et al., EMBO J. (1985) 4:761; transcription regulatory elements derived from the LTR of the Rous sarcoma virus, as described in Gorman et al., Proc. Nat'l Acad. Sci U.S.A. (1982) 79:6777; transcription regulatory elements derived from the LTR of human cytomegalovirus (CMV), as described in Boshart et al., Cell (1985) 41:521; hsp70 promoters, (Levy-Holtzman ,R. and I. Schechter (Biochim. Biophys. Acta (1995) 1263: 96-98) Presnail, J. K. and M. A. Hoy, (Exp. Appl. Acarol. (1994) 18: 301308)) and the like.

In many embodiments, the at least one transcriptionally active gene or module encodes a protein that has therapeutic activity for the multicellular organism, where such proteins include, but are not limited to: factor VIII, factor IX, β-globin, low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, omithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, interleukins, cytokines, small peptides etc, and the like. The above list of proteins refers to mammalian proteins, and in many embodiments human proteins, where the nucleotide and amino acid sequences of the above proteins are generally known to those of skill in the art.

In certain embodiments, the vector also includes at least one transcriptionally active gene or expression module that functions as a selectable marker. A variety of different genes have been employed as selectable markers, and the particular gene employed in the subject vectors as a selectable marker is chosen primarily as a matter of convenience. Known selectable marker genes include: the thymidine kinase gene, the dihydrofolate reductase gene, the xanthine-guanine phosporibosyl transferase gene, CAD, the adenosine deaminase gene, the asparagine synthetase gene, the antibiotic resistance genes, e.g. tet^{r}, amp^{r}, Cm^{r}or cat, kan^{r}or neo^{r}(aminoglycoside phosphotransferase genes), the hygromycin B phosphotransferase gene, genes whose expression provides for the presence of a detectable product, either directly or indirectly, e.g. β-galactosidase, GFP, and the like.

In addition to the above elements, the subject plasmid vectors also typically include a plasmid origin of replication. Representative plasmid origins of replication that may be present on the subject minimal plasmid vectors include, but are not limited to: ColE1 compatibility group origins like pUC and pBR322 oris, e.g., pMB1 ori, and p15A ori, etc.

An important feature of the subject minimal plasmid vectors employed in the subject methods is that they do not include bacterial plasmid sequences that would cause the plasmid vector to provide only transient, as opposed to persistent, expression. Expression is considered to be transient if expression is not persistent according to the guidelines provided above. Bacterial sequences that are to be avoided can readily be determined by those of skill in the art using the evaluation assays provided in the Experimental section , below.

The overall length of the minimal plasmid vector is sufficient to include the desired elements as described above, but not so long as to prevent or substantially inhibit to an unacceptable level the ability of the vector to enter the target cell upon system administration to the host. As such, the minimal plasmid vector is generally at least about 2 kb long, often at least about 4 kb long, usually at least about 6 kb long and more usually at least about 8 kb long, where the vector may be as long as 50 kb or longer, but in many embodiments does not exceed about 8 kb long and usually does not exceed about 10 kb long. In many embodiments, the length of the dsDNA vector ranges from about 1 to 10 kb, usually from about 3 to 8 kb, and more usually from about 4 to 6 kb.

The above described minimal plasmid vectors may be produced using any convenient protocol. The procedures of cleavage, plasmid construction, cell transformation and plasmid production involved in many protocols employed to prepare the subject vectors are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra.

A specific representative minimal plasmid vector of particular interest is a pUC derived minimal plasmid vector, and more specifically a pUC 18/19 derived vector. By pUC 18/19 derived vector is meant that the minimal plasmid vector includes a “backbone” domain that is a portion of the pUC 18/19 vector. The pUC 18/19 vector is well known in the art and whose map and sequence is publicly available on the ATCC website, as well as numerous other public sources. The portion of the pUC 18/19 vector which serves as the backbone of the minimal plasmid vector of these embodiments includes at least the ori domain, i.e., the pMB1 ori, and often also includes the Amp^{r}domain, but does not include the entire vector sequence. Of particular interest as the backbone in certain minimal plasmid vectors is the portion of the pUC 18/19 vector which is produced upon cleavage of the vector with AatII and AflIII, where the cleavage product includes both the pMB1 ori and the Amp^{r}domains. Specific representative vectors of this embodiment described in greater detail below include the pN series of vectors, i.e., pN, pN1, pN2, pN3, pN4, pN5. Also of interest are vectors that include just the ori domain of pUC 18/19, which ori domain may be linked to a different marker domain, e.g., Kan^{r}, as is found in pNKan.

Vector Administration

The subject methods find use in a variety of applications in which it is desired to introduce an exogenous nucleic acid into a target cell, and are particularly of interest where it is desired to expression a protein encoded by an expression cassette in a target cell. As mentioned above, a feature of the subject methods is that a minimal plasmid vector is systemically administered to a multicellular organism that includes the target cell, i.e., the cell into which introduction of the nucleic acid is desired. By multicellular organism is meant an organism that is not a single-celled organism. The multicellular organism to which the vector is administered is an organism that includes a plurality of cells and is not a single-celled precursor thereof. Multicellular organisms of interest include plants and animals, where animals are of particular interest. Animals of interest include vertebrates, where the vertebrate is a mammal in many embodiments. Mammals of interest include; rodents, e.g., mice, rats; livestock, e.g., pigs, horses, cows, etc., pets, e.g., dogs, cats; and primates, e.g., humans. As the subject methods involve administration of the vector directly to the multicellular organism, they are in vivo methods of introducing the exogenous nucleic acid into the target cell.

The route of administration of the vector to the multicellular organism depends on several parameters, including: the nature of the vectors that carry the system components, the nature of the delivery vehicle, the nature of the multicellular organism, and the like, where a common feature of the mode of administration is that it provides for in vivo delivery of the vector components to the target cell(s) via a systemic route. Of particular interest as systemic routes are vascular routes, by which the vector is introduced into the vascular system of the host, e.g., an artery or vein, where intravenous routes of administration are of particular interest in many embodiments.

Any suitable delivery vehicle may be employed, where the delivery vehicle is typically a pharmaceutical preparation that includes an effective amount of the minimal plasmid vector present in a pharmaceutically acceptable carrier, diluent and/or adjuvant. In certain embodiments, the minimal plasmid vector is administered in an aqueous delivery vehicle, e.g., a saline solution. As such, in many embodiments, the vector is administered intravascularly, e.g., intraarterially or intravenously, employing an aqueous based delivery vehicle, e.g., a saline solution.

The minimal plasmid vector is administered to the multicellular organism in an in vivo manner such that it is introduced into a target cell of the multicellular organism under conditions sufficient for expression of the nucleic acid present on the vector to occur. A feature of the subject methods is that they result in persistent expression of the nucleic acid present thereon, as opposed to transient expression, as indicated above. By persistent expression is meant that the expression of nucleic acid at a detectable level persists for an extended period of time, if not indefinitely, following administration of the subject vector. By extended period of time is meant at least 1 week, usually at least 2 months and more usually at least 6 months. By detectable level is meant that the expression of the nucleic acid is at a level such that one can detect the encoded protein in the mammal, e.g., in the serum of the mammal, at a level of at detectable levels at a therapeutic concentration. See e.g., the experimental section, supra. As compared to a control in which a pBluescript vector is employed, protein expression persists for a period of time that is at least about 2 fold, usually at least about 5 fold and more usually at least about 10 fold longer following the subject methods as compared to a control.

A feature of many embodiments of the subject methods is that the above described persistent expression is achieved without integration of the vector DNA into the target cell genome of the host. As such, the vector DNA introduced into the target cells does not integrate into, i.e., insert into, the target cell genome, i.e., one or more chromosomes of the target cell. In other words, the vector DNA introduced by the subject methods does not fuse with or become covalently attached to chromosomes present in the target cell into which it is introduced by the subject methods.

The particular dosage of vector that is administered to the multicellular organism in the subject methods varies depending on the nature of vector, the nature of the expression module and gene, the nature of the delivery vehicle and the like. Dosages can readily be determined empirically by those of skill in the art. For example, in mice where the vectors are intravenously administered in a saline solution vehicle, the amount of vector that is administered in many embodiments typically ranges from about 2 to 100 and usually from about 10 to 50 μg/mouse. The subject methods may be used to introduce nucleic acids of various sizes into the a target cell. Generally, the size of DNA that is inserted into a target cell using the subject methods ranges from about 1 to 12 kb, usually from about 3 to 10 kb, and sometimes from about 4 to 8 kb.

The subject methods may be employed to introduce a nucleic acid into a variety of different target cells. Target cells of interest include, but are not limited to: muscle, brain, endothelium, hepatic, and the like. Of particular interest in many embodiments is the use of the subject methods to introduce a nucleic acid into at least a hepatic cell of the host.

### Utility

The subject methods find use in a variety of applications in which the introduction of a nucleic acid into a target cell is desired. Applications in which the subject vectors and methods find use include: research applications, polypeptide synthesis applications and therapeutic applications. Each of these representative categories of applications is described separately below in greater detail.

Research Applications

Examples of research applications in which the subject methods of nucleic acid introduction find use include applications designed to characterize a particular gene. In such applications, the subject vector is employed to introduce and express a gene of interest in a target cell and the resultant effect of the inserted gene on the cell's phenotype is observed. In this manner, information about the gene's activity and the nature of the product encoded thereby can be deduced. One can also employ the subject methods to produce models in which overexpression and/or misexpression of a gene of interest is produced in a cell and the effects of this mutant expression pattern are observed.

Polypeptide Synthesis Applications

In addition to the above research applications, the subject methods also find use in the synthesis of polypeptides, e.g. proteins of interest. In such applications, a minimal plasmid vector that includes a gene encoding the polypeptide of interest in combination with requisite and/or desired expression regulatory sequences, e.g. promoters, etc., (i.e. an expression module) is introduced into the target cell, via in vivo administration to the multicellular organism in which the target cell resides, that is to serve as an expression host for expression of the polypeptide. Following in vivo administration, the multicellular organism, and targeted host cell present therein, is then maintained under conditions sufficient for expression of the integrated gene. The expressed protein is then harvested, and purified where desired, using any convenient protocol.

As such, the subject methods provide a means for at least enhancing the amount of a protein of interest in a multicellular organism. The term ‘at least enhance’ includes situations where the methods are employed to increase the amount of a protein in a multicellular organism where a certain initial amount of protein is present prior to in vivo administration of the vector. The term ‘at least enhance’ also includes those situations in which the multicellular organism includes substantially none of the protein prior to administration of the vector. By “at least enhance” is meant that the amount of the particular protein present in the host is increased by at least about 2 fold, usually by at least about 5 fold and more usually by at least about 10 fold. As the subject methods find use in at least enhancing the amount of a protein present in a multicellular organism, they find use in a variety of different applications, including agricultural applications, pharmaceutical preparation applications, and the like, as well as therapeutic applications, described in greater detail infra.

Therapeutic Applications

The subject methods also find use in therapeutic applications, in which the vectors are employed to introduce a therapeutic nucleic acid, e.g., gene, into a target cell, i.e., in gene therapy applications, to provide for persistent expression of the product encoded by the nucleic acid present on the vector. The subject vectors may be used to deliver a wide variety of therapeutic nucleic acids. Therapeutic nucleic acids of interest include-genes that replace defective genes in the target host cell, such as those responsible for genetic defect based diseased conditions; genes which have therapeutic utility in the treatment of cancer; and the like. Specific therapeutic genes for use in the treatment of genetic defect based disease conditions include genes encoding the following products: factor VIII, factor IX, β-globin, low-density lipoprotein receptor, adenosine deaminase, purine nucleoside phosphorylase, sphingomyelinase, glucocerebrosidase, cystic fibrosis transmembrane conductor regulator, α1-antitrypsin, CD-18, omithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, β-glucuronidase, α-L-iduronidase, galactose 1-phosphate uridyltransferase, and the like, where the particular coding sequence of the above proteins that is employed will generally be the coding sequence that is found naturally in the host being treated, i.e., human coding sequences are employed to treat human hosts. Cancer therapeutic genes that may be delivered via the subject methods include: genes that enhance the antitumor activity of lymphocytes, genes whose expression product enhances the immunogenicity of tumor cells, tumor suppressor genes, toxin genes, suicide genes, multiple-drug resistance genes, antisense sequences, and the like.

The subject methods also find use in the expression of RNA products, e.g., antisense RNA, ribozymes etc., as described in Lieber et al., “Elimination of hepatitis C virus RNA in infected human hepatocytes by adenovirus-mediated expression of ribozymes,” J Virol. (1996 December) 70(12):8782-91; Lieber et al., “Related Articles Adenovirus-mediated expression of ribozymes in mice,” J Virol. (1996 May) 70(5):3153-8; Tang et al., “Intravenous angiotensinogen antisense in AAV-based vector decreases hypertension,” Am J Physiol. (1999 December) 277(6 Pt 2):H2392-9; Horster et al. “Recombinant AAV-2 harboring gfp-antisense/ribozyme fusion sequences monitor transduction, gene expression, and show anti-HIV-1 efficacy, Gene Ther. (1999 July) 6(7):1231-8; and Phillips et al., “Prolonged reduction of high blood pressure with an in vivo, nonpathogenic, adeno-associated viral vector delivery of AT1-R mRNA antisense,” Hypertension. (1997 January) 29(1 Pt 2):374-80. As such, the subject methods can be used to deliver therapeutic RNA molecules, e.g., antisense, ribozyme, etc., into target cells of the host.

An important feature of the subject methods, as described supra, is that the subject methods may be used for in vivo gene therapy applications. By in vivo gene therapy applications is meant that the target cell or cells in which expression of the therapeutic gene is desired are not removed from the host prior to contact with the vector system. In contrast, the subject vectors are administered directly to the multicellular organism and are taken up by the target cells, following which expression of the gene in the target cell occurs. Another important feature is that the resultant expression is persistent and occurs without integration of the vector DNA into the target cell genome.

### Kits

Also provided by the subject invention are kits for use in practicing the subject methods of in vivo nucleic acid delivery to target cells, e.g., hepatic cells. The subject kits generally include the minimal plasmid vector, which vector may be present in an aqueous medium. The subject kits may further include an aqueous delivery vehicle, e.g. a buffered saline solution, etc. In addition, the kits may include one or more restriction endonucleases for use in transferring a nucleic acid into the vector. In the subject kits, the above components may be combined into a single aqueous composition for delivery into the host or separate as different or disparate compositions, e.g., in separate containers. Optionally, the kit may further include a vascular delivery means for delivering the aqueous composition to the host, e.g. a syringe etc., where the delivery means may or may not be pre-loaded with the aqueous composition.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g. a piece or pieces of paper on which the information is printed, in the packaging of the kit, in a package insert, etc. Yet another means would be a computer readable medium, e.g. diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

I. Vector Preparation

a. pN

pN carries a 1.8-kb AatII-AflIII fragment of pUC19, which includes a prokaryotic promoter, the beta-lactamase gene, and a terminal repeat of Tn3 transposon and ColE1 origin of replication. A NotI linker is inserted for easy cloning of a gene of interest.

b. pN5

pN5 carries a 1.8-kb AatII-AflIII fragment of pUC19 from which a dispensable terminal repeat sequence of Tn3 is removed. The prokaryotic beta-lactamase expression cassette and ColE1 origin of replication were independently amplified by PCR with a combination of Taq polymerase and Pfu polymerase, then these two PCR fragments were ligated to make pN5. There is a Not I site for cloning of a gene of interest.

c. pN1-4 are basically the same as pN except for the relative orientation of the prokaryotic beta-lactamase expression cassette to ColE1 origin of replication. The beta-lactamase expression cassette and ColE1 origin of replication were independently amplified by PCR with a combination of Taq polymerase and Pfu polymerase, then these two PCR fragments were ligated to make pN 1-4. There is a Not I site in each construct for cloning of a gene of interest.

d. pNkan

pNkan is a 1.9-kb plasmid containing an minimal prokaryotic aminoglycoside phosphotransferase gene expression cassette from Tn903 transposon, and ColE1 origin of replication. The Tn903 fragment also contains a part of a terminal repeat because the transcription termination signal is supposed to reside in the terminal repeat. The Tn903 fragment and ColE1 origin of replication were independently amplified by PCR with a combination of Taq polymerase and Pfu polymerase, then these two PCR fragments were ligated to make pNkan. There is a Not I site for cloning of a gene of interest.

e. pBS

pBS is pBluescript II KS(-) from Stratagene. A modified version of pBluescript KS(-), i.e., pBSFseIMCS , is also made. pBSFseIMCS carries additional multi-cloning sequences (FseI-PmeI-Sse83871-NotI-SwaI-StuI-FseI) between BamHI and XhoI sites in pBluescript II KS(-).

II. Expression Cassettes Tested

a. EF1α-hFIX

The expression cassette, EF11C-hFIX, is a human coagulation factor IX (hFIX)-expressing cassette driven by the human elongation factor α (EF1α) gene enhancer-promoter. This expression cassette is derived from pV4.1e-hFIX (Nakai et al., Blood (1998) 91: 4600), but a dispensable 1.3-kb Spel-MunI fragment was removed from the EF1α gene sequence.

b. TEF1α-hFIX

This expression cassette is derived from pT-EF1α-hFIX (Yant, et al., Nat. Genet. (2000) 25: 35). TEF1α-hFIX contains the EF1α-hFIX expression cassette carried by pV4.1α-hFIX and two terminal repeats of the Sleeping-Beauty transposon outside the cassette.

c. CM1

The expression cassette, CM1, is a hFIX-expressing cassette driven by an hybrid liver-specific enhance-promoter described by Miao et al. (Miao et al., Molecular Therapy (2000) 1:522). This expression cassette contains apolipoprotein E hepatic locus control region, the humanα1-antitrypsin gene promoter, hFIX cDNA containing a truncated intron A of the hFIX gene (hFIX minigene), and the bovine growth hormone gene polyadenylation signal.

III. Results

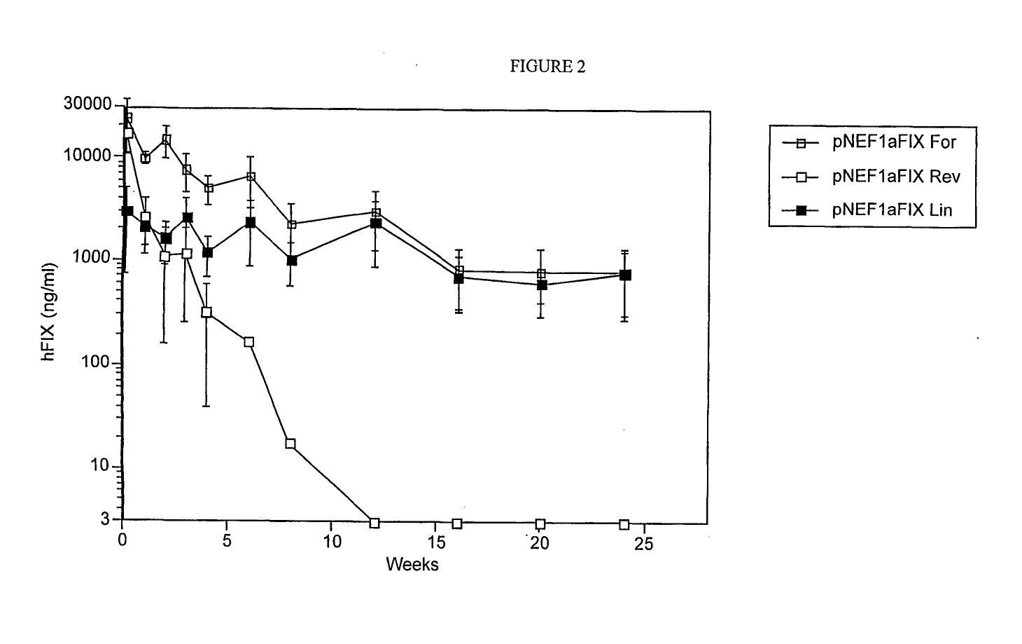

A. hFIX levels in mouse plasma following high-pressure tail vein injection of hFIX plasmid vectors. Six to eight-week old C57B1/6 mice received 25 μg of supercoiled closed circular plasmid, pNEF1αhFIX For, pNEF1αhFIX Rev, or linearized plasmid, pNEF1αhFIX Lin. pNEF1αhFIX For and pNEF1αhFIX Rev carry the same components (pN backbone and the EF1α-hFIX expression cassette) but the pN backbone is placed in different orientations, i.e., in the same orientation relative to the expression cassette for pNEF1αhFIX For while in the opposite orientation for pNEF1αhFIX Rev. pNEF1αhFIX Lin is the mixture of linearized pN backbone and EF1α-hFIX expression cassette. The results are shown in FIG. 2.

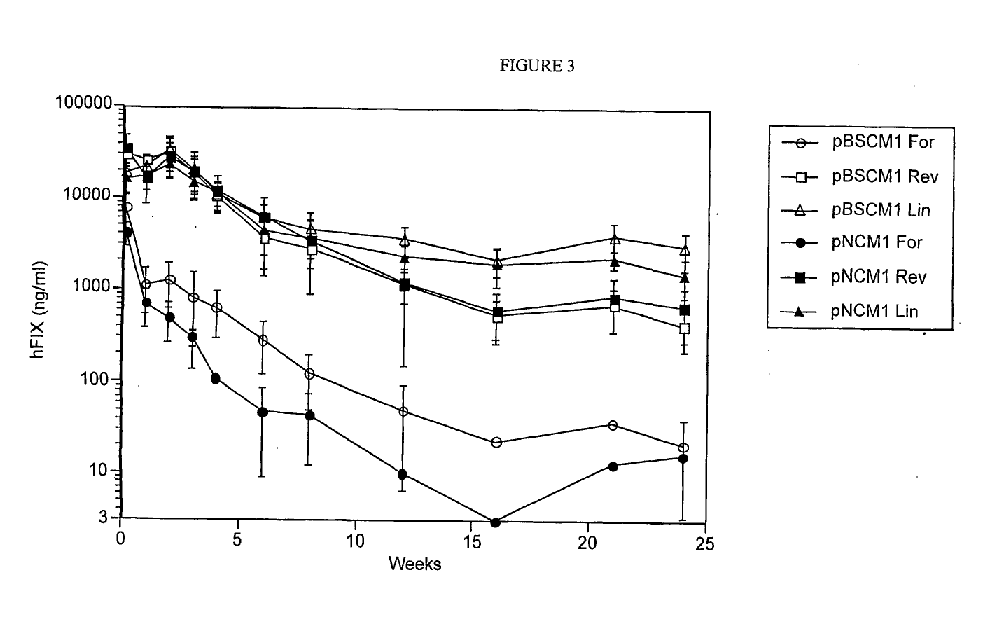

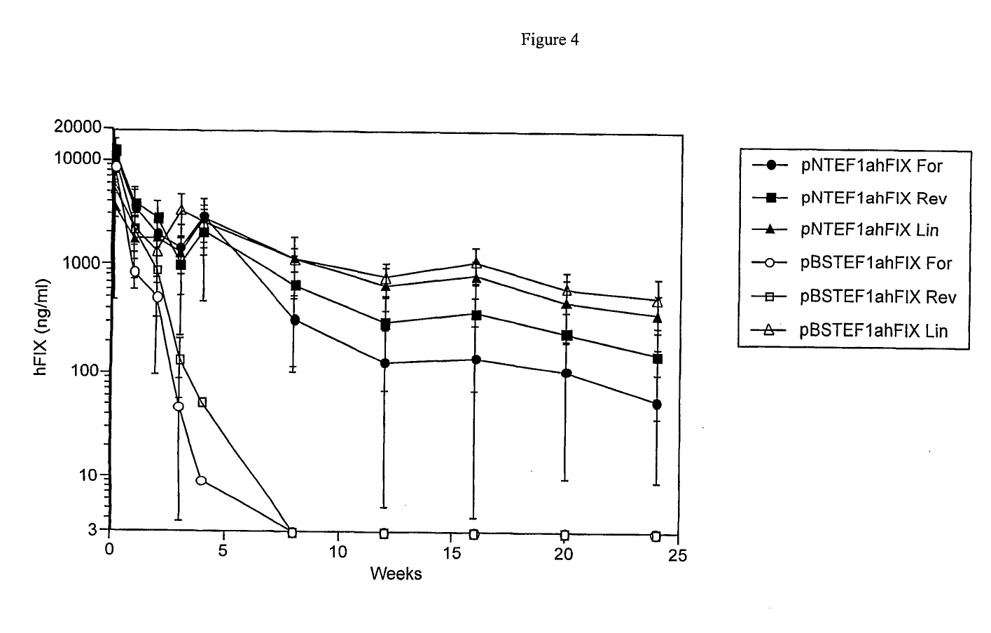

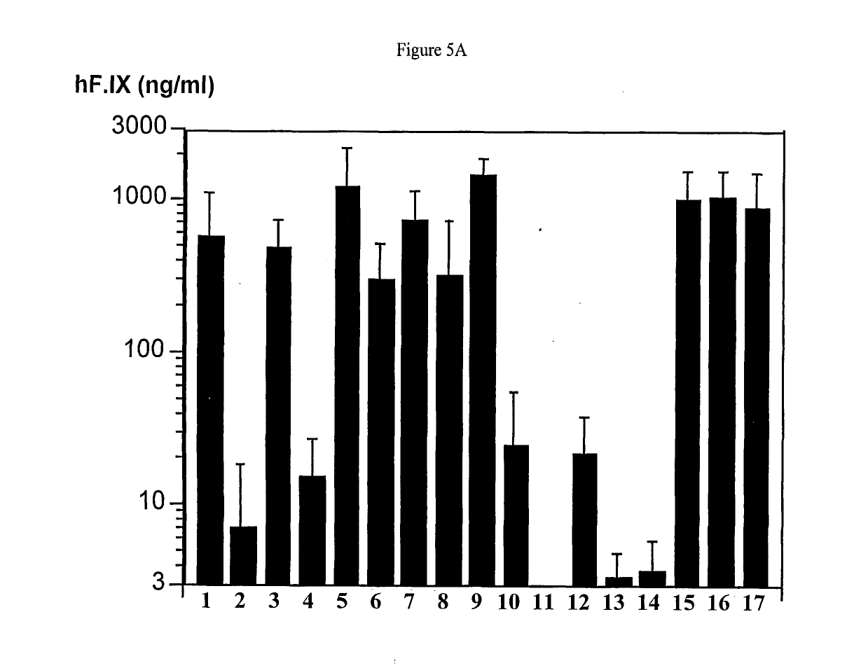

B. Comparison of the effect of pBS and pN backbones on persistent hFIX expression from mouse hepatocytes transduced by HFIX plasmid vectors. Six to eight-week old C57B1/6 mice received 25 μg (for pN constructs) or 30 μg (for pBS constructs) of HFIX plasmid vector by high-pressure tail vein injection, and plasma HFIX levels were followed. The results are shown in FIGS. 3 and 4.

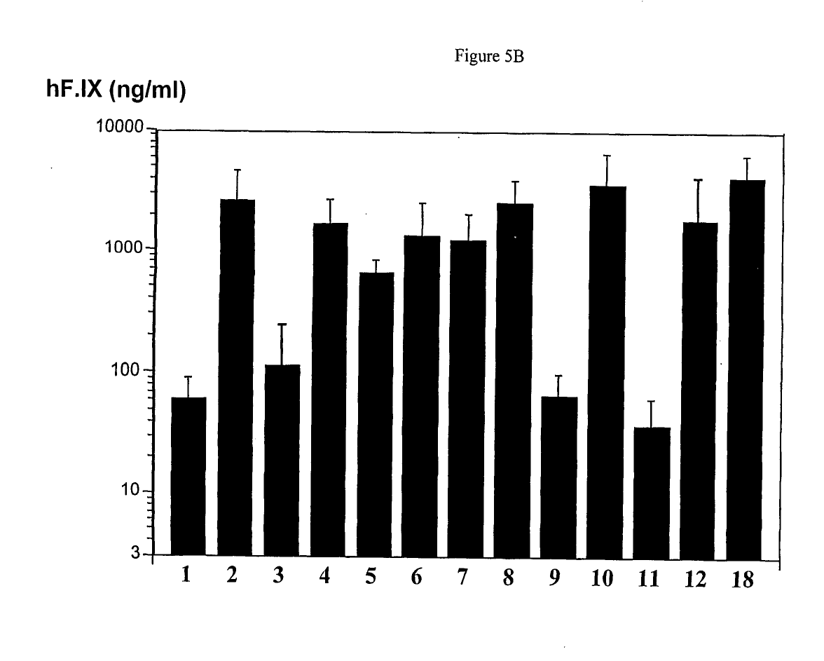

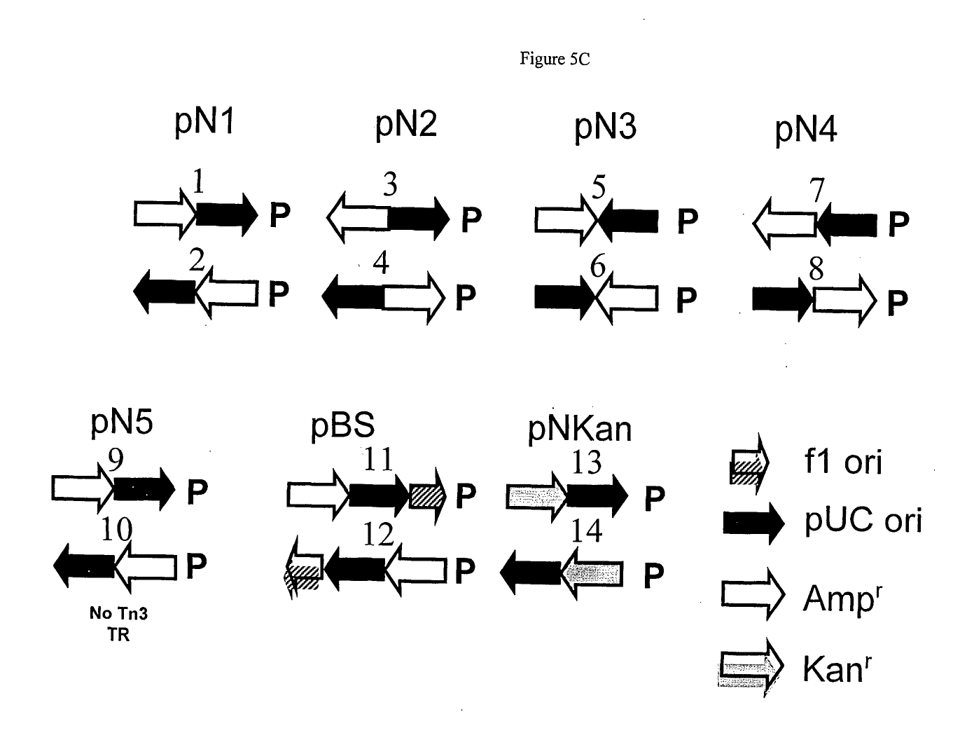

C. The experimental results suggested the plasmid ori is the element responsible for position and orientation-dependent inhibitory effect of plasmid backbone.. However, by changing the position or orientation of each element of minimal plasmid backbones, efficient and persistent transgene expression could be achieved, and orientation-dependent inhibitory effect of plasmid backbone could be minimized (see FIG. 5).

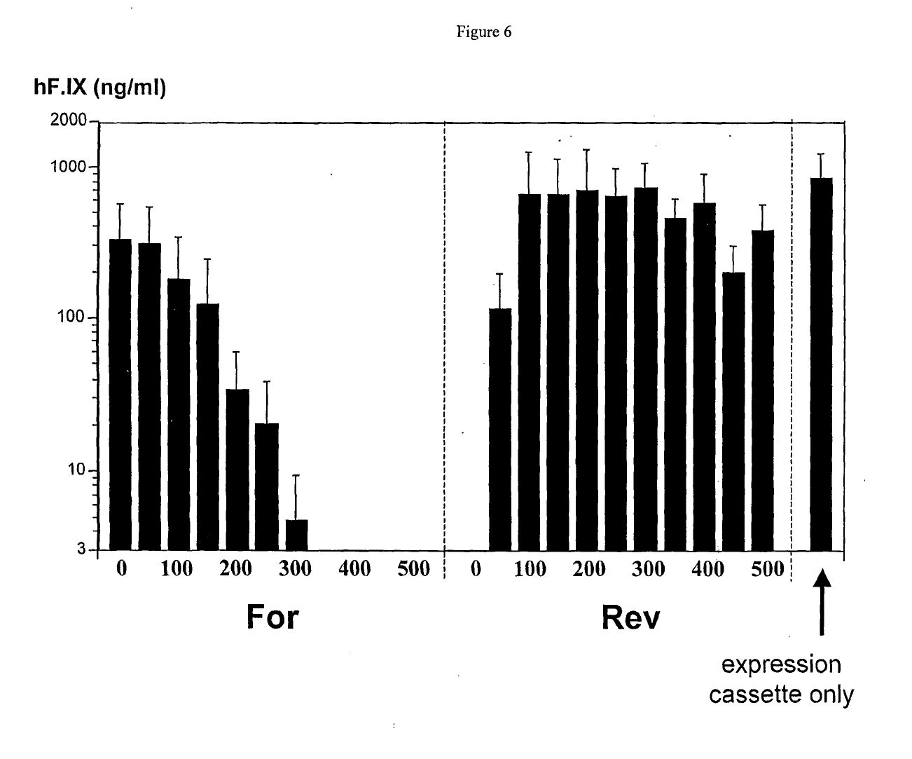

D. The size of the plasmid is an important factor for efficient and persistent transgene expression (see FIG. 6).

It is evident from the above results and discussion that an improved method of transferring a nucleic acid into a target cell is provided by the subject invention. Specifically, the subject invention provides for a highly efficient in vivo method for nucleic acid transfer which does not employ viral vectors and does not require target cell genome integration and yet provides for persistent high level gene expression and therefore provides many advantages over prior art methods of nucleic acid transfer. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes. of clarity of understanding, it is readily apparent to those of ordinary skill in the art in light of the teachings of this invention that certain changes and modifications may be made thereto without departing from the spirit or scope of the appended claims.

What is claimed is:

1. A method for introducing an expression cassette into a target cell of a vascularized multi-cellular organism in a manner such that the encoded protein of said expression cassette is persistently expressed in said target cell at a high level, said method comprising:
systemically administering to said vascularized multi-cellular organism a minimal plasmid vector comprising said expression cassette, wherein said minimal plasmid vector provides for persistent and high level expression in a manner that is substantially expression cassette sequence and direction independent;
to persistently express said expression cassette encoded protein at a high level in said target cell.

2. The method according to claim 1, wherein said administering is intravenous.

3. The method according to claim 1, wherein said vascularized multi-cellular organism is a mammal.

4. The method according to claim 1, wherein said minimal plasmid vector further comprises an antibiotic resistance gene.

5. The method according to claim 1, wherein said minimal plasmid vector further comprises a multiple cloning site.

6. The method according to claim 1, wherein said minimal plasmid vector further comprises a plasmid origin of replication.

7. The method according to claim 1, wherein said target cell is hepatic cell.

8. A method of expressing a protein in a target cell of a mammal, said method comprising:
intravenously administering to said mammal an aqueous formulation of a minimal plasmid vector comprising an expression cassette encoding said protein, wherein said minimal plasmid vector provides for persistent and high level expression in a manner that is substantially expression cassette sequence and direction independent;
whereby said expression cassette encoded protein is expressed in said target cell.

9. The method according to claim 8, wherein said minimal plasmid vector further comprises an antibiotic resistance gene.

10. The method according to claim 8, wherein said minimal plasmid vector further comprises a multiple cloning site.

11. The method according to claim 8, wherein said minimal plasmid vector further comprises a plasmid original of replication.

12. The method according to claim 8, wherein said target cell is a hepatic cell.

13. A method of persistently expressing a protein at a high level in a hepatic target cell of a mammal, said method comprising:
intravenously administering to said mammal an aqueous formulation of a minimal plasmid vector comprising an expression cassette encoding said protein, wherein said minimal plasmid vector provides for persistent and high level expression in a manner that is substantially expression cassette sequence and direction independent;
whereby said expression cassette encoded protein is persistently expressed at a high level in said hepatic target cell.

14. The method according to claim 13, wherein said minimal plasmid vector further comprises an antibiotic resistance gene.

15. The method according to claim 13, wherein said minimal plasmid vector further comprises a multiple cloning site.

16. The method according to claim 13, wherein said minimal plasmid vector further comprises a plasmid original of replication.

17. A minimal plasmid vector that provides for persistent and high level expression of an expression cassette present therein in a manner that is substantially expression cassette sequence and direction independent.

18. The minimal plasmid vector according to claim 17, wherein said vector further comprises a multiple cloning site.

19. The minimal plasmid vector according to claim 18, wherein said vector further comprises a plasmid origin of replication.

20. The minimal plasmid vector according to claim 17, wherein said vector further comprises an expression cassette.

21. A pharmaceutical composition comprising as an active agent a minimal plasmid vector that provides for persistent and high level expression of an expression cassette present therein in a manner that is substantially expression cassette sequence and direction independent together with a pharmaceutically acceptable carrier, diluent and/or adjuvant.

22. The composition of claim 21 for expression of a heterologous nucleic acid in a vascularized multi-cellular organism.

23. The composition of claim 21, which is administered systemically or locally.

24. The composition of claim 21, for gene therapy.

25. The composition of claim 21, for nucleic acid vaccination.

26. The use of a minimal plasmid vector that provides for persistent and high level expression of an expression cassette present therein in a manner that is substantially expression cassette sequence and direction independent for the manufacture of an agent for heterologous gene expression in a vascularized multi-cellular organism.
